(19) United States
(12) Patent Application Publication
(10) Pub. No.: US 2020/0093783 A1
(43) Pub. Date: Mar. 26, 2020

(54) Preparation and Anti-HepG2 Application for Urushiol-Based pH Response Micelle Encapsulating Paclitaxel

(71) Applicants: Nanjing Zhongsen Biological Technology Co., Ltd., Nanjing (CN); Institute of Chemical Industry of Forest Products, Chinese Academy of Forestry, Nanjing (CN)

(72) Inventors: Chengzhang Wang, Nanjing (CN); Zhiwen Qi, Nanjing (CN)

(73) Assignees: Nanjing Zhongsen Biological Technology Co., Ltd.; Institute of Chemical Industry of Forest Products, Chinese Academy of Forestry

(21) Appl. No.: 16/695,219

(22) Filed: Nov. 26, 2019

(30) Foreign Application Priority Data
Mar. 12, 2019 (CN) 201910191093.3

(51) Int. Cl. A61K 31/337; A61K 9/107; A61K 47/44

(52) U.S. Cl. CPC A61K 31/337; A61K 47/44; A61K 9/1075

(57) ABSTRACT

The present invention synthesizes a pH respond micelle such as BPAU-NH₂-Gal-PTX micelle which is composed of an urushiol hydrophobic alkyl chain through Michael addition. The experimental analysis of cytotoxicity in vitro and in vivo shows that BPAU-NH₂-Gal-PTX enters a HepG2 cell through endocytosis mediated by an ASGPR, releases urushiol and paclitaxel through the micelle, and inhibits the proliferation of the cell. A targeted BPAU-NH₂-Gal micelle has advantages such as high-efficient uptake, enhanced anti-tumor effect, favorable biocompatibility, etc. The present invention first applies the urushiol borate to a polymeric micelle of paclitaxel in vivo delivery, so that the urushiol and paclitaxel are loaded together on a micelle unit and used for being in combination with an anti-cancer synergistic therapy, which not only provides an idea for functional study of urushiol biochemical anti-tumor material, but also lays a foundation for the practical application of an urushiol medical micelle.

### TECHNICAL FIELD

The present invention belongs to the technical field of biological medicine, and relates to a preparation method and an anti-HepG2 application for an urushiol-based, micelle encapsulating paclitaxel; the urushiol-based micelle encapsulating paclitaxel is a kind of micelle nanocluster that introduces an forest bioactive urushiol derivative, has pH response to release the paclitaxel and exert tumor EPR (enhanced permeability and retention effect) effect, which can increase tumor therapeutic effect.

### BACKGROUND

Since the nano-carrier formed by self-assembly of an amphiphilic polymer has small 3D structure (10-200 nm), the nano-carrier can be enriched in a tumor site by the enhanced permeability and retention effect (EPR) of a solid tumor, and increases the drug concentration of the subacidity and high reducibility of the tumor site. It is noted that the functionalization of some galactosamine having a micelle encapsulating, paclitaxel results in considerable stagnation of a G2/M phase and a free drug, so that the cytotoxicity to the HepG2 cell is improved through the paclitaxel of delivery encapsulation in the cell. The particle size and the surface potential of the micelle are two important factors that influence the distribution of the micelle. The average particle size of a paclitaxel-loaded micelle is less than 200 nm, which matches the effect of the EPR.

The natural product polyethylene glycol has better biocompatibility and biodegradability, can be used as a hydrophilic segment to form a micelle shell, to make the micelle stable and extend the cycle time of micelle. For example, anti-colon cancer performance of curcumin is improved through MPEG-PLA micelle coating the curcumin, and since the pH value of the normal tissue is 7.4, and the acidity of tumor tissue such as lysosome and nucleosome is 56, the polymers such as MPEG-PLA-PHIS, etc. have been pH sensitive polymeric micelles as drug carriers. When the pH sensitive micelle is selectively enriched in a cancer cell through the EPR, effect, an acid environment is favorable for promoting the release of the drug.

Urushiol is an important natural product in East Asia, has favorable biological activity, and adjusts the synthesis of the DNA. The IC₅₀ (50% inhibiting concentration) of the urushiol to most of tumor cells is 4 μg/ml, and the anti-tumor. Activity is positively correlated with the saturation level of urushiol side chain. The nitrogen atoms are introduced into a molecular structure of the borate, which has important significance of increasing the hydrolytic stability of the borate. For an urushiol borate derivative, the hydrolysis rate thereof is closely related to the structure of an ester group and an internal structure. It is proved that an urushiol borate molecule is an ideal and stable, unit, and can be used as biocompatible polymeric micelle. In the method reported earlier, lauryl amine and tetradecylamine are respectively used as hydrophobic groups of MPEG-PBAE-C12 and MPEG-PBAE-C14. However, urushiol with a long-side alkane chain is taken as the hydrophobic group, and the lauryl amine and the tetradeclamine are replaced by the urushiol derivative as a hydrophobic unit.

### SUMMARY

The purpose of the present invention is to use an urushiol borate derivative as a micelle unit, and take amphiphilic diblock copolymer of BUDA, MPEG-NH₂ and 5-amino-1-pentanol as basic urushiol micelle units. The surface functionalization is conducted on a 13PAU-NH₂ micelle by adopting the galactosamine, so that the drug delivery is conducted on a targeted HepG2 cell ASGP-R. The urushiol-based polymeric micelle is generated through boric acid esterification reaction. Two kinds of drugs such as urushiol and paclitaxel have shown favorable biological activity of a synergistic anti-HepG2, which can be used as a kind of micelle with high-efficient synergistic function anti-HepG2 micelle. Three kinds of polymeric micelles such as BPAU. BPAU-NH₂ and BPAU-NH₂-Gal embedding PTX are assembled, and the stability, and drug delivery efficiency of the polymeric micelles in vivo and in vitro are characterized.

### DESCRIPTION OF DRAWINGS

FIG. 1 a transmission electron microscope photo of a micelle BPAU-NH₂-Gal, showing that the prepared micelle is evenly distributed and the morphology is, relatively rounding.

FIG. 2 a transmission electron microscope photo of a micelle BPAU-NH₂-Gal encapsulating paclitaxel, showing that the prepared micelle is evenly distributed and the morphology is, relatively rounding.

FIG. 3 is, a particle size distribution diagram of a micelle BPAU-NH₂-Gal.

FIG. 4 is a particle size distribution diagram of a micelle BPAU-NH₂-Gal encapsulating paclitaxel.

FIG. 5 shows the release of paclitaxel and urushiol in vivo by a drug-loaded micelle BPAU-NH₂-Gal-PTX in PBS.

FIG. 6 is the cytotoxicity of micelles such as PTX-13PAU-NH₂ and PTX-BPAU-NH₂-Gal to HepG2.

FIG. 7 is the cytotoxicity of micelles such as PTX-BPAU-NH₂, PTX-BPAU-NH₂-Gal, URU and BPAU to HepG2.

FIG. 8 is relative intensity of fluorescence of nano-drugs such as BPAU, BPAU-NH₂ and BPAU-NH₂-Gal embedding paclitaxel at the pH value of 4.5.

FIG. 9 is gross tumor volume of different groups of tumor-bearing mice after normal saline treatment for various days (a. shows normal saline used as blank control; b. shows a polymeric micelle BPAU: c. shows the polymeric micelle BPAU-NH₂; d. shows the polymeric micelle BPAU-NH₂-Gal).

FIG. 10 is a photo of a removed tumor for histological analysis on H&E dyeing of different processing groups under virtual microscopic observation ((200× vision) (a. shows normal saline used as blank control; b. shows paclitaxel; c. shows the polymeric micelle BPAU-NH₂; d. shows the polymeric micelle BPAU-NH₂-Gal).

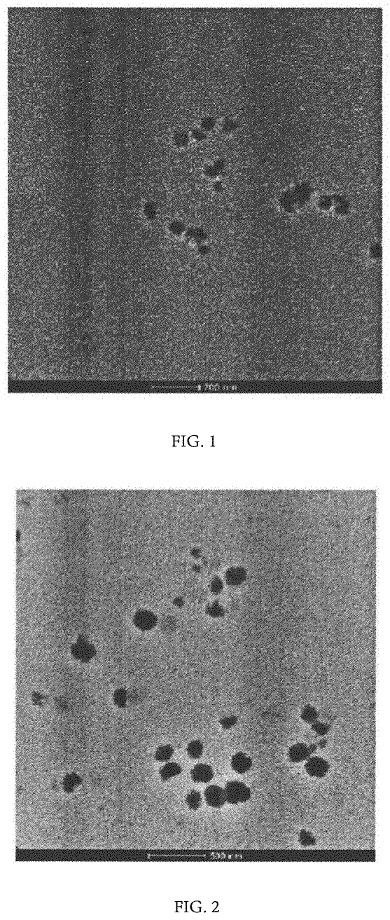

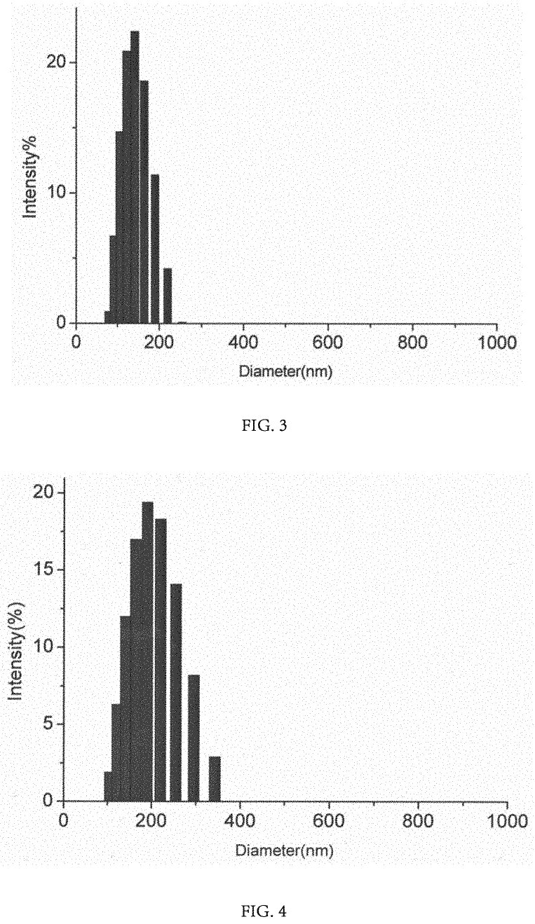

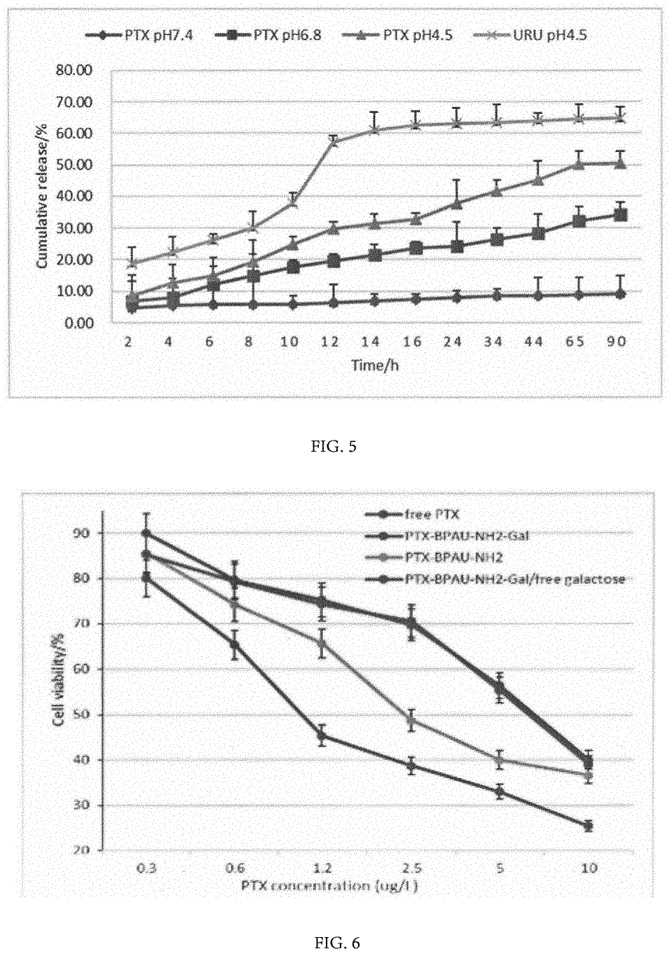

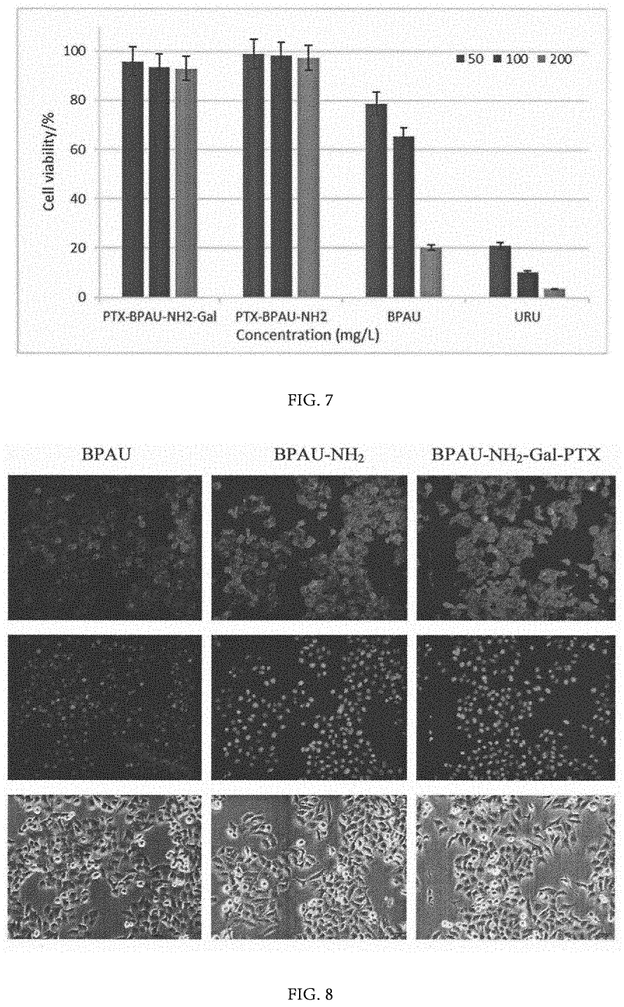

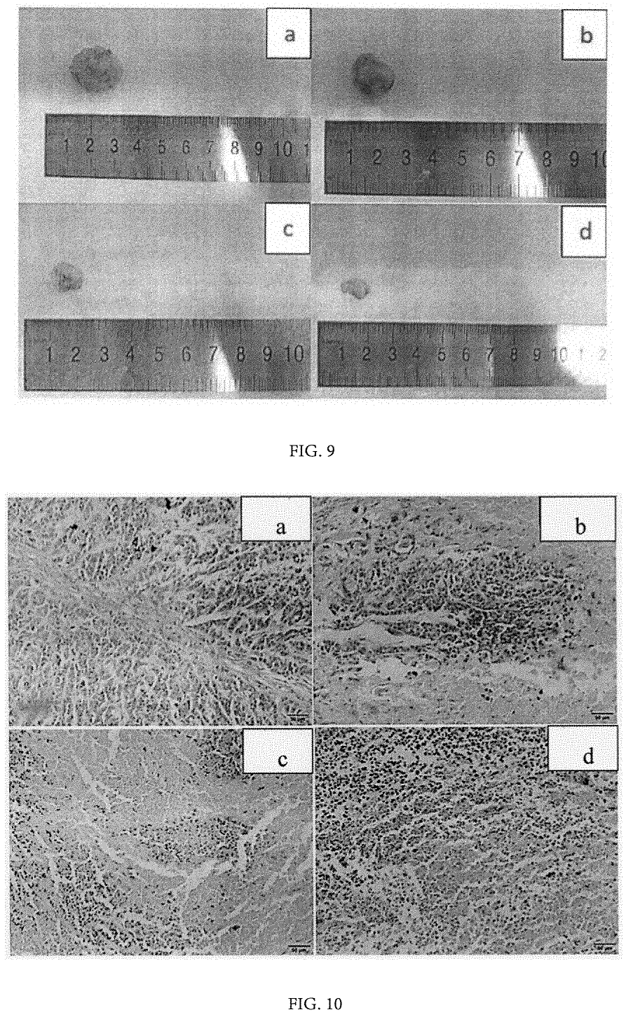

### DETAILED DESCRIPTION

The purpose of the present invention is realized by the following technical solution:

Synthesis of BPAU micelle: according to previous reports, MPEG-NH₂-PBAE is prepared. MPEG-NH₂ (0.08 eq., hydrophobic amine (0.7 eq.), 5-amino-1-pentanol (0.3 eq.) and BUDA (1.0 eq.) are weighed and dissolved in DMSO. Subsequently, the excessive 1, 3-Diamino-pentane (1.4 eq.) is added to an unreacted acrylate group, and then continuously stirred at 60° C. for one day.

Synthesis of BPAU-NH₂ micelle: hereafter, the URU-NH₂ (0.08 eq.) is added to the above mixture, and self-assembly is conducted through Michael addition reaction. Reaction polymerization is conducted on the mixture at 60° C. for 24 h, and the reaction mixture is diluted with 20 ml of dichloromethane and flushed with deionized water twice to remove the unreacted 1, 3-Diamino-pentane. An organic layer is dried with anhydrous magnesium sulfate for one night. After the magnesium sulphate is removed, concentration and spin drying are conducted, and finally drying is conducted under a vacuum condition for one night, to obtain a polymeric micelle.

Synthesis of BPAU-NH₂ micelle: the mixture of galactosamine (1.0 eq.), BPAU-NH₂ (1.0 eq.) and CDI (1.2 eq.) refluxes TEA (1.2 eq.) in MeCN for 1 hour. The acetonitrile is dropwise added to the reaction mixture, and after refluxing for 10 hours, a product is concentrated and washed with ethyl acetate. The coarse product is dialyzed from H₂O—H₃PO₄ (v/v=100:0.5).

2. Structure Determination

Instrument: Avance-400 Hz nuclear magnetic resonance spectrometer (Swiss Bruker corporation, internal standard TMS),

URU-NH₂: Umber liquid; R_{f}=0.75 (PE/EA=1:1); 1H NMR (400 MHz, CDCl3) δ 7.42 (d, J=3.06 Hz, 1H), 7.34 (s, 1H), 7.17 (m, 1H), 7.04 (d, J=2.80 Hz, 1H), 6.94 (t, J=3.86 Hz, 1H), 6.87 (d, J=2.85 Hz, 1H), 6.59 (s, 2H), 6.26 (t, J=8.81 Hz, 1H), 6.15 (s, 2H), 5.89 (t, J=2.65 Hz, 1H), 5.34-5.26 (m, 2H). 13C NMR (101 MHz, CDCl3) δ 148.13, 146.81, 145.74, 132.30, 131.08, 131.06, 129.93, 129.41, 129.22, 127.90, 126.85, 125.57, 124.30, 123.18, 122.33, 121.40, 119.24, 114.70, 114.08, 13.29. ESIMS m/z 449.4[C₂₇H₃₄BNO₂-NH₂]+.

BPAU/BUDA-(mPEG-NH₂)-(5-amino-1-pentanol): 1H NMR (400 MHz, CDC13) δ 6.71, 5.81, 4.42, 5.38, 5.11, 5.02, 4.97, 2.83, 2.79, 2.60, 2.03, 1.61, 1.32.13C NMR (101 MHz, CDCl3) δ 172.54, 70.43, 63.77, 53.53, 27.04, 23.39.

BPAU-NH₂/(BUDA-(mPEG-NH₂)-(5-amino-1-pentanol)-(URU-NH₂)): 1H NMR (400 MHz, CDCl3) δ 6.70, 6.62, 6.34, 5.97, 5.63, 5.61, 5.41, 4.99, 4.09, 2.80, 2.59, 2.27, 1.72, 1.26.13C NMR (101 MHz, CDCl3) δ 165.83, 143.40, 142.45, 129.37, 62.46, 58.62, 42.66, 40.71, 22.65.

BPAU-NH₂-Gal: 1H NMR (400 MHz, CDCl3) δ 6.72, 6.69, 6.38, 5.90, 5.65 (d), 5.60, 5.58, 5.41, 4.45, 4.00, 3.60 (m), 2.74, 2.70 (t), 2.32, 2.42, 1.70, 1.21.13C NMR (101 MHz, CDCl3) δ 164.36, 140.39, 141.22, 125.31, 97.63, 72.9, 70.5, 65.01, 63.65, 60.21, 55.21, 49.54, 40.01. 22.85.

1. A preparation and an anti-HepG2 application for an urushiol-based pH respond micelle encapsulating paclitaxel, characterized in that a BPAU and an urushiol borate URU-NH₂ are used to construct an amphiphilic molecular micelle. The particle size of the micelle BPAU-NH₂-Gal encapsulating paclitaxel is about 195 nm.

2. The synthetic urushiol-based micelle according to claim 1, characterized in that the synthetic urushiol-based micelle has the following structure features, and the chemical structure is as follows:

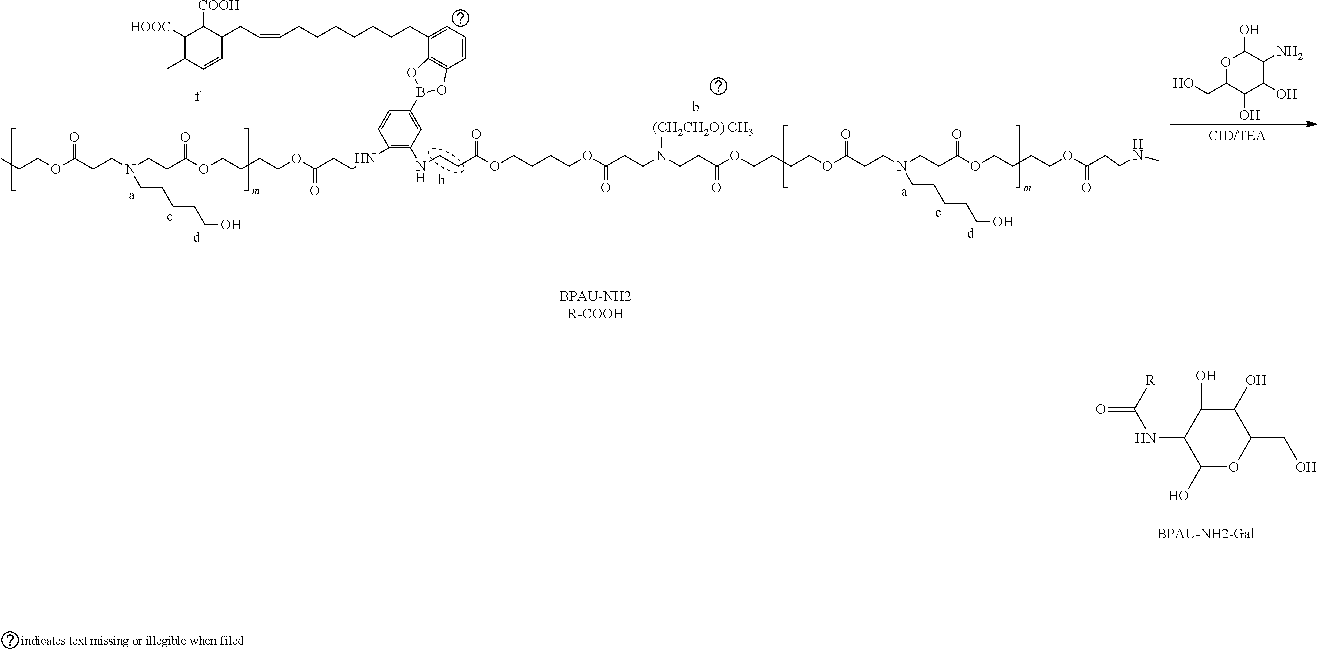

3. The preparation and an anti-HepG2 application for an urushiol-based pH respond micelle encapsulating paclitaxel according to claim 1, characterized by comprising the following synthesis steps:
(1) Weighing MPEG-NH₂ (0.08 eq.), hydrophobic amine (0.7 equivalent weight), 5-amino-1-pentanol (0.3 eq.) and BUDA (1.0 eq.) and dissolving in DMSO.
(2) Adding the excessive 1,3-Diamino-pentane (1.4 eq.) to an unreacted acrylate group, and then continuously stirring at the temperature of 60° C. for one day.
(3) Adding the URU-NH₂ (0.08 eq.) to the above mixture, and conducting self-assembly through Michael addition reaction.
(4) Conducting reaction polymerization on the mixture at the temperature of 60′C for 24 h, diluting the reaction mixture with 20 ml of dichloromethane, and flushing with deionized water twice to remove the unreacted 1,3-Diamino-pentane.
(5) Drying an organic layer with anhydrous magnesium sulfate for one night and after the magnesium sulphate is removed, conducting concentration and spin drying, and finally drying under a vacuum condition for one night, to obtain a polymeric micelle.
(6) Refluxing TEA (1.2 eq.) by the mixture of galactosamine (1.0 eq.), BPAU-NH₂ (1.0 eq.) and CDI (1.2 eq.) in acetonitrile for 1 hour.
(7) Dropwise adding the acetonitrile to the reaction mixture, and after refluxing for 10 hours, concentrating a product and washing with ethyl acetate.
(8) Dialyzing the coarse product from H₂O—H₃PO₄ (v/v=100:0.5).

4. The synthetic urushiol-based micelle according to claim 2, characterized by comprising MPEG (methoxypolyethylene glycol Mn=5000) and polymine (PEI 25K, MW=25 KD) and dialysis membrane POR6 (MWCO 5000, 8000).

5. The synthetic urushiol-based micelle according to claim 2, characterized in that the zeta potential of the BPAU-NH₂-Gal-PTX is +30 mV.

6. The synthetic urushiol-based micelle according to claim 2, characterized in that the particle size of the BPAU-NH₂-Gal-PTX is about 195 nm, and PDI value is 1.44.

7. The synthetic urushiol-based micelle according to claim 2, characterized in that the percentage of the BPAU-NH₂-Gal-PTX encapsulating paclitaxel is 99.5% or so.

8. The synthetic urushiol-based micelle according to claim 2, characterized in that when the pH value of the BPAU-NH₂-Gal-PTX micelle is 4.5, the drug release rates of the paclitaxel and the urushiol are respectively 64.1% and 49.5%.

9. A preparation and anti-HepG2 application for urushiol-based pH respond micelle encapsulating paclitaxel, characterized in that the experimental analysis of cytotoxicity in vitro and in vivo shows that BPAU-NH₂-Gal-PTX enters a HepG2 cell through endocytosis mediated by an ASGPR, releases the urushiol and the paclitaxel through the micelle, and inhibits the HepG2 cell proliferation.

10. The synthetic urushiol-based micelle according to claim 8, characterized in that the BPAU-NH₂-Gal-PTX results in gradual reduction of the cell proportions in an S period and G2/M period of the HepG2, thus resulting in the death of cancer cells.

11. The synthetic urushiol-based micelle according to claim 8, characterized in that the urushiol and the paclitaxel released by the BPAU-NH₂-Gal-PTX can enter a HepG2 cell and inhibit the HepG2 cell growth.
